(19) United States
(12) Patent Application Publication
(10) Pub. No.: US 2008/0241002 A1
(43) Pub. Date: Oct. 2, 2008

(54) SYSTEM AND METHOD FOR REPROCESSING A DEVICE HAVING INTERNAL PASSAGEWAYS

(75) Inventors: Craig Weber, Chalfont, PA (US); Douglas Palovick, Philadelphia, PA (US); Michael Patzek, Quakertown, PA (US)

Correspondence Address: CAESAR, RIVISE, BERNSTEIN,;COHEN & POKOTILOW, LTD., 11TH FLOOR, SEVEN PENN CENTER, 1635 MARKET STREET, PHILADELPHIA, PA, 19103-2212, US

(73) Assignee: CUSTOM ULTRASONICS, INC., Buckingham, PA (US)

(21) Appl. No.: 12/059,051

(22) Filed: Mar. 31, 2008

Related U.S. Application Data
(60) Provisional application No. 60/908,728, filed on Mar. 29, 2007.

(51) Int. Cl. A61L 2/24; A61L 2/18; H04L 9/32

(52) U.S. Cl. 422/109; 422/292; 726/19

(57) ABSTRACT

A system for reprocessing contaminated medical instruments includes at least one reprocessing unit, the reprocessing unit being adapted to perform the reprocessing of at least one of a predetermined type of contaminated medical instrument. The system also includes an electronic controller including a user input device, an electronic processor, associated memory, and an operating system capable of being run on the processor. The controller is operably coupled to the reprocessing unit to enable control of the operation of the reprocessing unit. A control program for the controller for disposition in the memory is provided. The control program establishes at least one protocol of processing steps for effecting the reprocessing of the medical instrument in the reprocessing unit. A monitor provides a visual display of various operating conditions of the system.

### BACKGROUND OF THE INVENTION

This invention relates generally to a system for the reprocessing of a contaminated device having internal passageways before such a device is reused in a clean environment. The term “reprocessing,” as used herein constitutes the washing, disinfecting, sterilizing and/or pasteurizing of such a device. The term “device” as used herein constitutes any devices having internal passageways that require such reprocessing, including, but not limited to, medical instruments and medical devices. The terms “medical instrument” and “medical device” are understood to constitute devices having one passageway or a plurality of passageways, including, but not limited to endoscopes, colonoscopes, and other flexible and rigid medical instruments.

Some automated systems for reprocessing devices having internal passageways for reuse are generally available and are commonly relied upon. For example, systems for reprocessing medical instruments having passageways are used by hospitals to safeguard patients and hospital employees from exposure to infection and cross-contamination. Such systems are manufactured by several different companies including, Custom Ultrasonics, Inc., of Ivyland, Pa., the assignee of the present invention and application. For example there are reprocessing units in the prior art adapted for cleaning, disinfecting and sterilizing flexible scopes, e.g., upper and lower gastrointestinal scopes, colonoscopes and duodescopes.

Prior art reprocessing systems, suitable in particular for reprocessing medical instruments, operate in accordance with a predetermined protocol of reprocessing steps. The protocol is based upon the specific cleaning requirements of the particular instruments being cleaned. The reprocessing steps are precisely timed and sequenced in order to assure optimal results, based upon the correct combination of water temperature, detergent and chemical agents. Thus, parameters such as wash and rinse cycle time, chemical immersion cycle time and water temperature and pressure were preset by the reprocessing unit manufacturer and could not be altered by an end user of the system. U.S. Pat. No. 5,761,069, issued to Weber, et. al. teaches a system for cleaning medical instruments having a database of protocols corresponding to differing medical instruments for permitting a user to load and execute the protocol corresponding to the instrument being reprocessed.

An exemplary protocol for cleaning a medical instrument could include the following reprocessing steps, after the instrument has been placed in the cleaning basin of the reprocessing unit: (1) wash the internal and external surfaces of the instrument with a measured detergent-water mixture for a preset period of time; (2) activate ultrasonic crystals while washing; (3) drain the detergent-water mixture after the wash cycle is completed; (4) after draining, rinse the internal and external surfaces of the instrument with water at a preset temperature for a preset period of time; (5) introduce and circulate disinfectant over and through the instrument for a preset period of time; (6) drain the disinfectant from the wash basin; and (7) after draining of the disinfectant is complete, rinse the instrument with water; and (8) re-rinse the instrument with water.

One current reprocessing system uses a computer that runs on a DOS system. A user may activate such reprocessing system simply by turning the system on and directing the reprocessing system to execute a particular reprocessing protocol. Although this may be convenient for the user, it does not prevent an unauthorized user from accessing and using the system. Additionally, since the system's computer does not recognize which user is accessing the system, there is no way to track which user operated the system at a particular time. Although current systems are largely automated, there are instances in which human error can play a role in inadequate or ineffective reprocessing of a medical instrument. Therefore, there is a need for a reprocessing system which can only be activated and operated upon a user's entering of information, e.g., a password, into the computer, which uniquely identifies the specific user. Moreover, concerns of security and accountability give rise to a need for a system which can store data identifying which user ran which reprocessing cycle on a particular instrument.

Current reprocessing systems are not adaptable to be linked into a local area network, such as that in a hospital. There is a need for data associated with the running of a reprocessing cycle to be transferred over a network, so that such data may be easily accessed by other people, such as a nurse administrator. Such data might include, for example, the name of the user of the reprocessing system, the identity of the specific instrument(s) reprocessed during a particular reprocessing cycle, the name of the patient(s) on which the instrument(s) was used and the name of the doctor(s) who used the instrument(s). In the unfortunate event that a patient is infected by one of the reprocessed instruments, it would be helpful for information regarding the reprocessing sequence of that instrument to be readily available over the network. This would improve patient care since those users who do not run reprocessing cycles properly could be held accountable.

When a user of a current reprocessing system is having trouble using the system or if the system appears to be malfunctioning, the user often contacts the manufacturer and/or distributor of the system by telephone to obtain technical assistance. When this is done, the user must verbally describe the problem and what he/she sees on the display screen of the system. In some cases, this process can be inefficient and unnecessarily time consuming. There is therefore a need for the manufacturer and/or distributor to have remote access to the particular system, so that the manufacturer and/or distributor could view the visual display on the system's monitor and control the system, thereby more efficiently and effectively assisting the user.

At times, the system configuration settings of reprocessing systems are accessed in order to reconfigure system parameters. This task is generally not done by medical facility staff, but rather, by representatives of the manufacturer and/or distributor of the system. In order to ensure that persons other than manufacturer and/or distributor representatives do not have access to system configuration settings (one concern being that the system may be improperly reconfigured so as to ineffectively reprocess instruments), current systems generally have a password for such access, intended to be known only by certain manufacturer and/or distributor personnel, e.g., representatives. For example, one such system currently has a permanent three-digit password with which one can gain access to system configuration settings. However, a permanent password, or even one that is only changed occasionally, may at some point be learned by those not intended to have access to the system configuration settings. There is a need, therefore, for greater security to prevent unintended users from having access to system configuration settings.

Current systems generally employ a liquid disinfectant (or liquid chemical germicide) to be used in the reprocessing of instrument(s). These systems have compartments or reservoirs for storage of the disinfectant so that it can be used upon initiating a reprocessing sequence. Some liquid disinfectants must be maintained within a particular temperature range, or else they may lose their effective properties. When the system is shut down, e.g., at night or on weekends, the first user to initiate the system currently has no way of knowing whether the disinfectant stored within a compartment of the system has been consistently maintained at an appropriate temperature. It is therefore the general practice to simply dispose of the disinfectant stored within the system after the system has been shut down. Disinfectant can be expensive and therefore there is a need to prevent any unnecessary waste of disinfectant stored within compartments of reprocessing systems.

There is also a need for a reprocessing system with software capable of threading, i.e., directing more than one function of the reprocessing system at one time.

### SUMMARY OF THE INVENTION

A system for reprocessing contaminated medical instruments includes at least one reprocessing unit, the reprocessing unit being adapted to perform the reprocessing of at least one of a predetermined type of contaminated medical instrument. The system also includes an electronic controller including a user input device, an electronic processor, associated memory, and an operating system capable of being run on the processor. The controller is operably coupled to the reprocessing unit to enable control of the operation of the reprocessing unit. A control program for the controller for disposition in the memory is provided. The control program establishes at least one protocol of processing steps for effecting the reprocessing of the medical instrument in the reprocessing unit. A monitor provides a visual display of various operating conditions of the system.

### BRIEF DESCRIPTION OF SEVERAL VIEWS OF THE DRAWINGS

The invention will be described in conjunction with the following drawings in which like reference numerals designate like elements and wherein:

FIG. 1 is an elevational view of a reprocessing unit suitable for use with the present invention.

FIG. 2 shows a top view of the reprocessing basin of the reprocessing unit of FIG. 2 including a device to be reprocessed.

FIG. 3 show a flow chart representation of a process for use with the system and method of the invention.

### DETAILED DESCRIPTION OF THE INVENTION

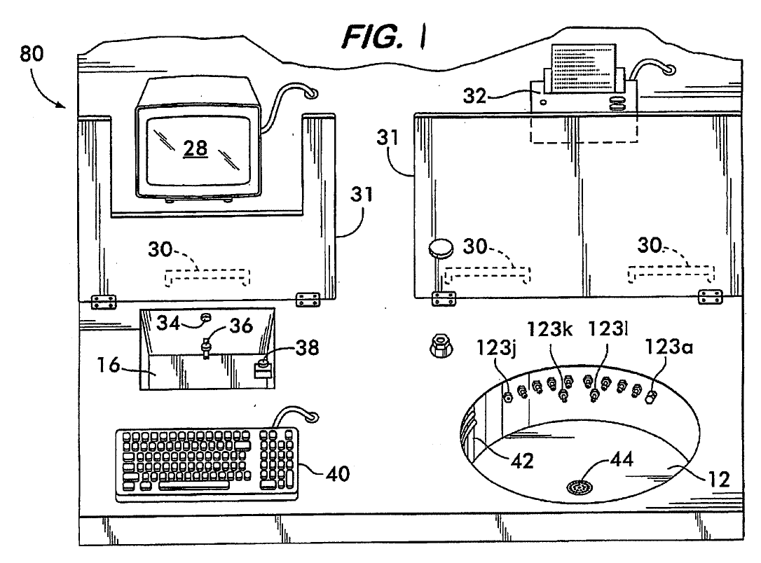

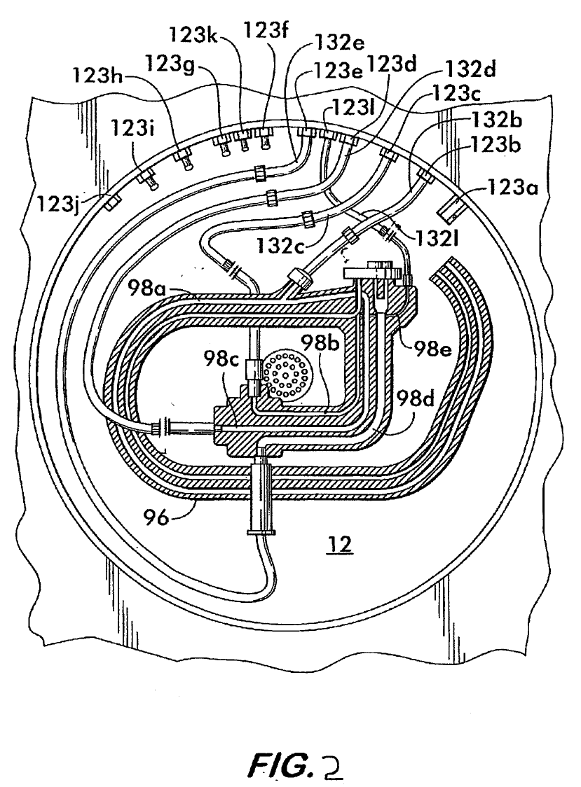

Referring now to FIGS. 1, 2, there is shown a reprocessing system 80 suitable for use with the present invention, and a view of a reprocessing basin 12 (a.k.a. processing chamber) within the reprocessing system 80. Custom Ultrasonics, Inc., of Ivyland, Pa., the assignee of the present invention and application, is the manufacturer of the System 83 Plus and MiniFlex, both of which are embodiments of the system 80 of the present invention.

The reprocessing basin 12 holds a device 96 having internal passageways 98a-e for reprocessing of the device 96 by the reprocessing system 80. In a preferred embodiment of the invention, the device 96 being reprocessed by the reprocessing system 80 can be a medical instrument 96. In particular, the system and method of the invention are well suited for application to medical instruments including flexible scopes such as endoscopes that are used for upper and lower gastrointestinal studies, bronchoscopes, cystoscopes, and ENT, urology and gynecology flexible endoscopes.

The reprocessing system 80 includes a keyboard 40, a monitor 28, a printer 32, and an associated personal computer (not shown) for permitting a user of the reprocessing system 80 to communicate with and control the reprocessing system 80. The reservoir 16 of the reprocessing system 80 includes the sensors 34, 36, 38 for controlling devices such as a heater, a pump and a vacuum device (not shown) in order to protect against failure conditions such as overflow conditions in the reservoir 16. A removable door 42 within the reprocessing basin 12 covers additional sensors (not shown) for providing further operational capability and safety protection during the operation of the reprocessing system 80. The door stops 30 are provided to stop the motion of the rotatable doors 31 covering the reservoir 16 and the reprocessing basin 12 when they are opened.

In a preferred embodiment, the reprocessing basin 12 can hold more than one device 96 upon a mesh for reprocessing of the internal passageways 98a-e thereof according to conventional reprocessing protocols. The reprocessing system 80 is adapted to provide fluid flows of differing pressures to the device 96 or devices 96 being reprocessed when the internal passageways 98a-e have differing diameters. The reprocessing system 80 is adapted to perform the multi-pressure reprocessing operations using a single pump (not shown), and to provide an indication of an obstruction in any of the internal passageways 98a-e of the device or devices 96 as described in more detail below. The single pump of the reprocessing system 80 can be a diaphragm pump, an oscillating pump, or any other type of pump known to those skilled in the art.

The reprocessing basin 12 includes the supply ports 123a-1 that can be selectively used to apply fluids at different fluid flow rates to the medical instruments 96 for reprocessing of the medical instruments 96. For example, the supply port 123j can be capped and reserved for use when needed. The supply port 123a can be used to blow off a fluid flow which is unusable due to difficulty in regulating and measuring their flow rates, as described in more detail below. In this example, at least the supply ports 123a-1 that are not capped or blown off can be vented into the reprocessing basin 12 or coupled to the internal passageways 98a-e of a medical instrument 96 as needed.

For example, an internal biopsy passageway 98a of the medical instrument 96 can be coupled to the supply port 123b by way of the tubing segment 132b, and an internal water channel passageway 98b of the medical instrument 96 can be coupled to the supply port 123c by way of the tubing segment 132c. The internal passageway 98c can be coupled to the supply port 123d by way of the tubing segment 132d, and the internal suction passageway 98d can be coupled to the supply port 123e by way of the tubing segment 132e. The internal elevator water channel passageway 98e can be coupled to the supply port 123l by way of the tubing segment 132l. Typical diameters for some of the passageways 98a-e can be 0.508 millimeters to 4.8 millimeters.

In one embodiment of the present invention (not shown), the computer of the reprocessing system 80 has stored within its memory data identifying at least one potential user of the system 80. Any and all potential users of the reprocessing system 80 are assigned a unique username and password to access the system 80. The system cannot be activated and a reprocessing sequence cannot be run without a user entering his/her username and password. Additionally, when a user enters a username and password, the computer or another electronic data storage device records the identity of the user who activated the system 80 and/or ran a reprocessing sequence. In this way, a record is maintained as to which user ran which reprocessing sequence. Although entering of a username and password is a preferred way to identify which user ran which sequence, there are many other possibilities as to how this may be achieved. For example, a user may be assigned a user identification card which has a magnetic strip, a bar code, a RFID tag or a RFID reader, or biometric data, which may be read by an input device operably connected to the computer. One advantage to requiring a user to identify himself/herself prior to accessing the system 80 is for security purposes, i.e., to ensure that only those persons intended to have access to the system 80 have access. Another advantage is that users who do not operate the system 80 properly may be held accountable. This improves patient service because patients may be at risk of contracting an infectious disease as a result of a medical device used on the patient which was not properly reprocessed.

In a related embodiment of the present invention (not shown), in addition to user identification data being recordable on an electronic data storage device, other data related to the reprocessing of the instruments may also be recorded onto the electronic data storage device. Such data may include data identifying the specific instrument that underwent reprocessing, data identifying the patient on which the instrument was used, data identifying the physician who conducted a procedure with the instrument, data regarding the temperature of the disinfectant used during reprocessing and data regarding the type of disinfectant used during reprocessing.

In another embodiment of the present invention (not shown), the computer of the reprocessing system 80 is part of a local area network, which allows the computer to transfer data associated with the reprocessing of instruments to other computers within the network. For example, the data generated in conjunction with the running of a reprocessing sequence may be transferred via the network to the computer of a nurse administrator or another hospital employee.

In another embodiment of the present invention (not shown), the computer of the reprocessing system 80 is accessible and operable by a remote user, such as the manufacturer and/or distributor of the system 80. In the event that a user is having difficulty using the system 80 or the system 80 appears to be malfunctioning, the manufacturer and/or distributor may remotely access the computer of the system 80 for purposes of troubleshooting and assisting the user.

In another embodiment of the present invention (not shown) the system's computer has system configuration settings stored in its memory. The settings store parameters of reprocessing protocols, for example, wash and rinse cycle time, chemical immersion cycle time and water temperature and pressure. If these parameters are improperly altered, it may render a reprocessing protocol ineffective, thereby detracting from the integrity of the system 80 as programmed and configured by its manufacturer and/or distributor. Therefore, in one embodiment of the present invention, the system configuration settings may be accessed only by entering a special password into the computer. It is intended that the password should not be known by users of the system 80, but rather, preferably by representatives of the manufacturer and/or distributor. Moreover, the password is changed periodically in order to prevent its dissemination to unintended users. In a preferred embodiment, the password is changed daily. For example, the manufacturer and/or distributor of the system 80 may actively change the password. Alternatively, the manufacturer and/or distributor of the system 80 would have access to an algorithm-generated password. In either case, if a representative of the manufacturer and/or distributor is at the site of a particular system 80, the representative may contact the manufacturer and/or distributor in order to obtain the appropriate password for that particular system 80 at that particular time. Upon obtaining the appropriate password, the representative may access the system configuration settings in order to make any desired changes. It is also preferred that the password not be short in length, but rather, it should be several characters long, e.g., twelve characters.

In another embodiment of the present invention (not shown) the computer of the system 80 provides the user with threading capabilities, i.e., to allow the user to operate more than one function of the system 80 at one time.

In another embodiment of the present invention (not shown) the system 80 further includes a compartment for storage of liquid disinfectant used for reprocessing medical instruments. Disinfectants often must be stored within a particular temperature range, or else they may lose their effectiveness. Therefore the compartment includes a temperature sensor to measure the temperature of the disinfectant. The compartment further includes a temperature regulator, e.g., a heater, to maintain the temperature of the disinfectant at a desired level. In one embodiment, measurements from the temperature sensor are periodically communicated to and stored in the computer of the system 80, or alternatively to another electronic data storage device. Temperature sensor measurements would preferably be communicated every 0.2 seconds to 10 minutes, and even more preferably every 1 to 5 seconds. A record of the temperature of the disinfectant could thereby be maintained, to ensure that the temperature was adequate at all times for the particular disinfectant. Additionally, the temperature regulator and sensor would continue to function even when the system 80 is not activated, is in “sleep mode”, when the monitor 28 displays a “screen saver”, and/or when the system is otherwise off (preferably, the system 80 is left on 24 hours a day, 7 days a week). In this way, even when the system 80 is inactive, e.g., during nights and weekends, users would be assured that the disinfectant in the compartment was maintained at an appropriate temperature at all times. This would obviate the need to dispose of the disinfectant before running a new reprocessing sequence.

In another embodiment of the present invention (not shown) the computer of the system 80 monitors and controls the temperature of disinfectant and water used during the reprocessing of a medical instrument.

In yet another embodiment of the present invention, upon completion of a reprocessing sequence, the printer 32 would automatically print information pertinent to the operations of the system 80, or any other information stored on the computer's memory. Such information may relate to one or more of the following reprocessing parameters: date and time of initiating the system 80, model and serial number of the medical instrument(s) which underwent reprocessing, physician name, patient name, technician name, detergent name, disinfectant name, time the reprocessing sequence started, wash time, rinse time, disinfectant time, temperature of the disinfectant, first rinse time, second rinse time, third rinse time, time the reprocessing sequence was completed and total time duration of the complete reprocessing sequence. In a related embodiment, in the event the printer 32 malfunctions or there is not a proper connection between the printer 32 and the computer, the problem would be communicated to the user, e.g., in the form of a dialogue box on the screen of the monitor 28. Failure to document and print-out these reprocessing parameters and include this documentation in the patient's medical file may hinder an investigation in the event an infection or outbreak is identified. Thus, optimally, the system 80 would be unable to run a reprocessing sequence unless and until such information, or some of such information is entered into the computer.

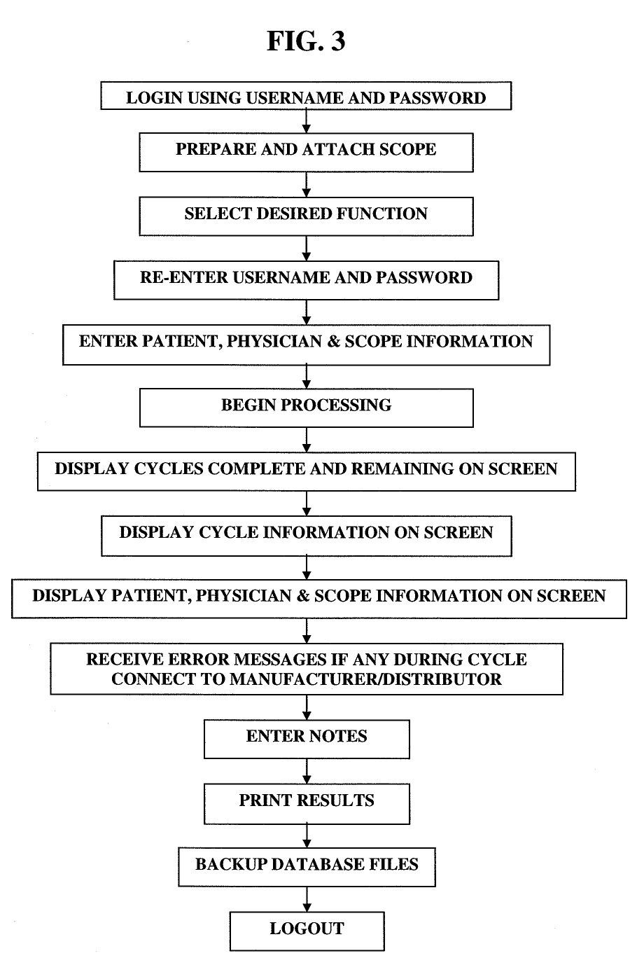

Referring now to FIG. 3, there is shown a flow chart representation of a process for use with the system and method of the invention. To perform this process a user can enter a username and a password into the computer of system 80 for the purpose of authentication. Accordingly, the user can view a main control screen, and use a main control screen to access to operate the system 80. In addition to providing access to the system 80 the main control screen can offer the user access to options such as obtaining reports, going into a night mode, logging off or shutting down. In a preferred embodiment the system is connected to a local area network, such as a hospital network, and to the internet.

The user can then prepare and attach the scope to be reprocessed by the system 80. Those skilled in the art will understand that the scope to be processed should be wiped down and that the insertion tube should be wiped down to remove lubricant and gross contaminants without exerting excessive pressure on bending rubber. The scope can be tested for leaks. Air, water and suction buttons can be removed and an appropriately sized cleaning brush can be passed through the suction/biopsy channel exiting at the distal tip and at the connector. The brush tip can be cleaned and removed. The user can clean the suction button housing and select and attach all appropriate instrument adapters for processing. A check can be made that all adapters are secure and the liquid can flow thorough all tubing.

The user can then select the desired functions. For example the user can select wash, disinfect or purge function. Before the system 80 performs the requested function on the attached scope, a second entering of a username and a password can be required. Furthermore, additional entering of usernames and passwords can be required at any time during the process of FIG. 3. This requirement can help to identify which user initiates each process. It also helps to prevent or remedy any errors during the process. For example, if a user tries to empty disinfectant from the system 80, adjust any parameters of the process, or start or stop a cycle during the process of FIG. 3 a new entry of a username and password can be required. This can help protect the user, the system 80, the scope, the hospital and the patient.

Additionally, an embodiment made available to administrators, charge nurses and bio-med staff for checking and fixing medical equipment can include additional screens to provide additional functionality beyond the functionality available to other users. The additional functionality can include, for example, functions for adding or deleting users and physicians.

Furthermore, any changes to the configuration of the system 80 can be restricted to the manufacturer or distributor of the system using usernames and passwords. Accordingly, the manufacturer or distributor can enter a user name and password at a remote location and change the configuration of the process as needed. The software controlling the operations of the system 80 can be modified or updated by the manufacturer or distributor from the remote location. The user can be notified prior to such updating and given the option of permitting the update to continue. For example, an option to perform a wash only function can be enabled or disabled from the remoter location. Additionally, any of the operations of the system 80 can be performed from the remote location. The username and password used by the manufacturer or distributor can be changed periodically in order to enhance security.

Any required modifications and updating of the system 80 can be performed automatically on a periodic basis or on an as-needed basis. In one embodiment selected error detection algorithms performed by the system 80 can result in error messages to the user and to the manufacturer or distributor in order to permit the manufacturer or distributor to assist the user in correcting the error. The manufacturer or distributor can view operation of the system 80 from the remote location in response to automatically transmitted error messages or call for assistance from the user in order to further assist in correcting the errors. The manufacturer or distributor can also run diagnostic algorithms from the remote location.

Operating system threading capabilities can be used to permit the manufacturer or distributor to monitor the activities of each individual machine bay within the system 80, including the inputs, the outputs and the temperature of any elements of the system 80. For example, the potency and temperature of the disinfectant can be monitored. The monitored parameters can all be monitored on a recurring basis, for example every three seconds. The results of all monitoring operations can be saved by the user or by the manufacturer or distributor. The results can be stored either on the system 80 computer or on a server located at a site operated by the manufacturer or distributor.

The user can enter patient, physician, and scope information relevant to the reprocessing operation into the system 80. As previously described, an individual entry of a username and password can be required in order to implement the entry of the patient or physician data. In a preferred embodiment all such information for a period of time, for example, for a period of a day, can be entered at one time. Patient information and physician information can then be adjusted as necessary during the period of time if needed. Thus, for example, if it is determined that a scheduled patient appointment is canceled the scope reserved for the patient can be removed.

When the system 80 and the user are ready the reprocessing of the scope can begin, with or without a further entry of a username and password. During reprocessing, the system 80 can provide displays to the user and/or the remote manufacturer or distributor. For example, the system 80 can display information such as the number of cycles completed, the number of cycles remaining, the percentage of the total reprocessing time, and the input and output flows of all fluids used during the reprocessing. The displays can indicate the operation of the ultrasonics, pumps such as drain pumps, drain valves, water valves, oscillating pumps, reservoir pumps, reservoir returns, residual drains, and detergent pumps. The monitored and displayed information can include the automated injection of alcohol. Additionally, patient, physician and scope information can be displayed. The display of relevant information allows staff to identify instruments and perform other operations without touching and opening the door of the system 80. For example, the user can check the model of the scope the within the system 80 by viewing the display or obtaining a printout. Thus, the user can obtain the information without incurring any risk of splashing or contamination.

In one preferred embodiment the user can be permitted to enter notes at any time during the reprocessing operations. Notes, messages, or other information can be printed out by the user or by the manufacturer or distributor. The information can be printed out at the system 80 or at the location of the server of the manufacturer or distributor. The notes, monitored information, or any other information, can be saved in the system 80 database or the manufacturer or distributor server database. Furthermore, the information can be made available as part of reports on the reprocessing session. Operation of the process of FIG. 3 can be terminated or returned to the beginning in order to perform further reprocessing.

It will be understood by those skilled in the art that a client program running on a remote computer with encrypted data transmissions can permit an approved remote user, such as a facility manager, a unit manager, a nurse manager, bio-med personnel, etc., to produce any desired charts and reports based on data stored within the system 80 or at the server operated by the manufacturer. This can include remote administration and adding, editing and deletion of, but not limited to, endoscopes, physicians, employees and users. Product information, industry alerts, and marketing updates can also be received by the client program. The client program can contain, but is not limited to, up to date manuals, product brochures, parts, price listings, etc.

The client program, back up database data, and any other reports and data may be transmitted over an encrypted connection. They may also be saved to a rewritable CD which is local to the computer running the client. Additionally, the system and method of the invention permits identification and logon by way of RFID, Wi-Fi based RFID technology, scanning employee cards or bar codes, and/or biometric based technologies.

The channel monitoring performed by the invention can be completely integrated to permit every monitored channel to be controlled and monitored by the software implementing the process of FIG. 3. Therefore, obstructions and/or blockages in the instrument channels, adapters, and inline filters can be monitored. The monitored information can be logged at any remote location, any location within the local area network, or at any location connected to the internet. Furthermore, determinations of flow level below or above a predetermined range can be made from any location. The determinations can be performed using RFID technology, and they can be performed whether a scope adapter is connected or unconnected to the scope. They can also be performed whether the scope adapter is connected or unconnected to the system 80.

Any or all of the various aforementioned embodiments may be combined to form further embodiments of the present invention. While the invention has been described in detail and with reference to specific embodiments thereof, it will be apparent to one skilled in the art that various changes and modifications can be made therein without departing from the spirit and scope thereof.

What is claimed is:

1. A system for reprocessing contaminated medical instruments, said system comprising:
a. at least one reprocessing unit, said reprocessing unit being adapted to perform the reprocessing of at least one of a predetermined type of contaminated medical instrument;
b. an electronic controller including a user input device, electronic processor, associated memory, and an operating system capable of being run on said processor, said controller being operably coupled to said reprocessing unit to enable control of the operation of said reprocessing unit;
c. a control program for said controller for disposition in said memory, said control program establishing at least one protocol of processing steps for effecting the reprocessing of the medical instrument(s) in said reprocessing unit; and
d. a monitor for providing a visual display of various operating conditions of said system.

2. The system of claim 1, wherein said operating system is in a Windows format.

3. The system of claim 1, wherein said memory is adapted for storing data uniquely identifying at least one potential user of said system.

4. The system of claim 3, wherein data identifying at least one potential user of said system is stored in said memory.

5. The system of claim 4 further comprising an electronic data storage device adapted to electronically record information related to the reprocessing of said medical instrument(s).

6. The system of claim 5 wherein said electronic data storage device is an internal hard drive, external hard drive, compact disc or floppy disk.

7. The system of claim 5 wherein said information related to the reprocessing of said medical instrument(s) includes one or more of the following: data identifying the user of said system, data identifying the specific instrument(s) that underwent reprocessing, data identifying the patient on which said instrument(s) was used, data identifying the physician who conducted a procedure with said instrument(s), data regarding temperature of disinfectant used during reprocessing and data regarding the type of disinfectant used during reprocessing.

8. The system of claim 4, wherein said system can be activated only upon communication from said user to said controller, whereby said communication uniquely associates said user with said data identifying said user stored in said memory.

9. The system of claim 4, wherein said user can run a protocol of processing steps only upon communication from said user to said controller, whereby said communication uniquely associates said user with said data identifying said user that is stored in said memory.

10. The system of claims 9 wherein said communication is effectuated by entering information into the controller with said user input device.

11. The system of claim 10 wherein said communication is in the form of entering a username/and or password uniquely associated with said user and wherein said input device is selected from the group consisting of a computer keyboard, a computer mouse, a touch-activated monitor, and a joystick.

12. The system of claim 10, wherein said communication is in the form of entering into said user input device information encoded onto a magnetic strip and wherein said user input device is a magnetic strip reader.

13. The system of claim 10, wherein said communication is in the form of entering information into said user input device using a RFID tag wherein said user input device is a RFID reader.

14. The system of claim 10, wherein said communication is in the form of entering information into said user input device using a RFID reader wherein said user input device is a RFID tag.

15. The system of claim 10, wherein said communication is in the form of entering biometric information into said user input device.

16. The system of claim 10, wherein said communication is in the form of entering bar code information and wherein said user input device is a bar code reader.

17. The system of claim 5, wherein said system is part of a local area network, whereby said information related to the reprocessing of said medical instrument(s) can be transmitted over said network.

18. The system of claim 1, wherein said electronic controller is accessible and operable by a remote user.

19. The system of claim 18, wherein said remote user is the manufacturer and/or distributor of said system.

20. The system of claim 1 wherein system configuration settings are stored on said memory, said settings establishing operating parameters of said system.

21. The system of claim 20 wherein said system configuration settings are accessible only upon entering of a special password into said user input device.

22. The system of claim 21 wherein said special password changes periodically.

23. The system of claim 22 wherein said special password changes daily.

24. The system of claim 22 wherein said special password is obtainable only from a manufacturer and/or distributor of said system.

25. A method of adjusting said system configuration settings of said system of claim 24 whereby a representative of said manufacturer and/or distributor obtains said special password from said manufacturer and/or distributor and enters said special password into said user input device thereby gaining access to said system configuration settings upon which said representative makes at least one adjustment to said system configuration settings.

26. The system of claim 22 wherein said special password is actively changed by a manufacturer and/or distributor of said system.

27. The system of claim 22 wherein said special password is automatically changed as a result of a preprogrammed algorithm which generates passwords.

28. The system of claim 1, said electronic controller having threading capabilities.

29. The system of claim 1 further comprising a compartment for storage of disinfectant used for reprocessing said medical instrument(s).

30. The system of claim 29 further comprising a temperature sensor within said compartment, said temperature sensor adaptable to measure the temperature of said disinfectant within said compartment and a temperature regulator to maintain the temperature of said disinfectant at a desired level.

31. The system of claim 30 further comprising an electronic data storage device adapted to electronically record the temperature, at periodic intervals of time, of said disinfectant.

32. The system of claim 31, said periodic intervals of time being between 0.2 seconds and 10 minutes.

33. The system of claim 31, wherein said temperature sensor continues to measure the temperature of said disinfectant and said temperature regulator continues to maintain the temperature of said disinfectant at a desired level, even when said system is not activated.

34. The system of claim 33 wherein said temperature sensor continues to measure the temperature of said disinfectant and said temperature regulator continues to maintain the temperature of said disinfectant at a desired level, even when said system is in “sleep mode”.

35. The system of claim 33 wherein said temperature sensor continues to measure the temperature of said disinfectant and said temperature regulator continues to maintain the temperature of said disinfectant at a desired level, even when said monitor is displaying a “screen saver”.

36. The system of claim 33 wherein said temperature sensor continues to measure the temperature of said disinfectant and said temperature regulator continues to maintain the temperature of said disinfectant at a desired level, even when said system is otherwise off.

37. The system of claim 1 further comprising a printer operably coupled to said electronic controller, said printer being adaptable to print information pertinent to the operations of said system or any other information stored on said memory.

38. The system of claim 37 wherein said information relates to one or more of the following reprocessing parameters: date and time of initiating said system, model and serial number of said medical instrument, physician name, patient name, technician name, detergent name, disinfectant name, time reprocessing sequence started, wash time, rinse time, disinfectant time, temperature of disinfectant, first rinse time, second rinse time, third rinse time, time process completed and total time duration of the complete reprocessing sequence.

39. The system of claim 38, wherein some or all of said information is automatically printed upon completion of a reprocessing sequence.

40. The system of claim 39, whereby the user is signaled in the event the printer malfunctions or has an improper connection to said electronic controller.

41. The system of claim 2, wherein said operating system is in a Windows XP format.

42. The system of claim 1, wherein an authentication is required for operation of said reprocessing unit.

43. The system of claim 42, wherein a further plural authentication is required for operation of said reprocessing unit.

44. The system of claim 43, wherein said further authentication is required for performing a predetermined reprocessing unit function.

45. The system of claim 1, further comprising a display for displaying reprocessing unit data.

46. The system of claim 1, further comprising a printer for printing reprocessing unit data.

47. The system of claim 46, further comprising a printout of an indication of a model identification of said medical instrument wherein said model identification can be determined without opening said reprocessing unit.

48. The system of claim 1, further comprising a system disposed at a remote location for reconfiguring said reprocessing unit from said remote location.

49. The system of claim 48, wherein an authentication is required for permitting said reconfiguring.

50. The system of claim 48, wherein said reconfiguring comprises editing said control program.

51. The system of claim 48, wherein said reconfiguring comprises adding or deleting a reprocessing unit function.

52. The system of claim 1, further comprising a system disposed at a remote location for monitoring said reprocessing unit from said remote location.

53. The system of claim 52, wherein said system disposed at said remote location assists in correcting errors in the operation of said reprocessing unit.

54. The system of claim 52, wherein said system disposed at said remote location determines the potency of a disinfectant located at said reprocessing unit.
